(19) United States
(12) Patent Application Publication
(10) Pub. No.: US 2008/0009950 A1
(43) Pub. Date: Jan. 10, 2008

(54) Prosthetic Knee

(76) Inventor: Rodney lan Walter Richardson, South Yarra (AU)

Correspondence Address: BAKER BOTTS L.L.P., 30 ROCKEFELLER PLAZA, 44TH FLOOR, NEW YORK, NY, 10112-4498, US

(21) Appl. No.: 11/776,291

(22) Filed: Jul. 11, 2007

Related U.S. Application Data
(63) Continuation of application No. PCT/AU05/01974, filed on Dec. 22, 2005.

(30) Foreign Application Priority Data
Jan. 12, 2005 (AU) 2005900132

(51) Int. Cl. A61F 2/38

(52) U.S. Cl. 623/20.29; 623/20.35; 623/20.33

(57) ABSTRACT

A prosthetic knee for replacing the knee of a patient including: (a) a femoral component being coupable to a resected distal end of a femur of patient; (b) a tibial component being couplable to a resected proximal end of a tibia of the patient; and (c) an insert, wherein the femoral component and the tibial component articulate by way of the insert arrangable therebetween, and a curved articular surface of the insert is adapted to pivot with respect to a corresponding curved articular surface of the tibial component.

### TECHNICAL FIELD OF THE INVENTION

The present invention relates to a prosthetic knee.

### BACKGROUND OF THE INVENTION

Disease or trauma that affect the articular surfaces of a knee can be treated by surgically replacing the ends of bones with prosthetic femoral and tibial implants. The femoral and tibial implants typically articulate by way of an insert arranged therebetween. The process of replacing the ends of bones in this manner is known as a total knee replacement.

Knee joint prostheses can be classified into two basic types. The first type, referred to as “stabilised” prosthesis, has hinge or ball type joints used as substitutes for the anatomical knee joint. In this type of knee joint, the movement of the joint is constrained by a hinge pin or ball and socket. The stabilised knee joint is useful where little reliance can be placed on the surrounding tendons and ligaments to stabilise the joint. However, unlike the anatomical joint, stabilised knee joints of this type permit little, if any, anterior-posterior translation, lateral angulation, or rotation.

In the second type of knee joint prosthesis, referred to as condylar surface prostheses, the corresponding bearing surfaces on the femur and tibia are replaced by analogously shaped and positioned prosthetic bearing surfaces. Condylar surface prosthesis joints rely upon the surrounding tendons and ligaments to hold the femoral and tibial portions of the joint together and to impart stability to the joint during movement. Prosthetic joints of this type have previously been used with some success. However, prosthetic joints of this type may not adequately simulate femoral roll back. For example, they may not permit posterior/anterior translation of the femoral bearing surface with respect to the tibial bearing surface during flexion/extension. Further, known condylar surface prostheses may not permit rotation of the femoral bearing surface with respect to the tibial bearing surface during flexion/extension.

Metal alloys have previously been used for the femoral and tibial components of prosthetic knee joints and polyethelene has been used as a material for the insert arranged therebetween. The bearing surfaces used in any such hard on soft articulation may create wear debris which is a major cause of osteolysis and implant failure. Lack of lubrication on the articulating surfaces of a prosthetic joint may also lead to joint failure. To minimise wear and subsequent failure, patients with prosthetic knees are often restricted in activity to that of low demand on the prosthetic device. Younger patients sometimes delay having surgery due to the short lifespan of artificial joints. While it may by possible to overcome these difficulties by using harder wearing biomaterials, the design of artificial knee joints may not necessarily facilitate use of such materials.

It is generally desirable to overcome or ameliorate one or more of the above mentioned difficulties, or at least provide a useful alternative.

### SUMMARY OF THE INVENTION

In accordance with one aspect of the present invention, there is provided a prosthetic knee for replacing a knee of a patient including:
(a) a femoral component being couplable to a resected distal end of a femur of the patient;
(b) a tibial component being couplable to a resected proximal end of a tibia of the patient; and
(c) an insert,
   wherein the femoral component and the tibial component articulate by way of the insert arrangable therebetween, and a curved articular surface of the insert is adapted to pivot with respect to a corresponding curved articular surface of the tibial component.

Preferably, said curved articular surface of the tibial component is adapted to facilitate translation of the insert with respect to the tibial component.

Preferably, the curved articular surface of the tibial component is convex and the curved articular surface of the insert is concave, and said concave articular surface is adapted to at least partially receive said convex articular surface.

In accordance with another aspect of the present invention, there is provided a femoral component for a condylar surface prosthetic knee of a patient, including:
(a) a non-articulating surface being couplable to a resected distal end of a femur of the patient;
(b) a lateral condyle articular surface with a radius of curvature, R₁, for engagement with an insert of the prosthetic knee; and
(c) a medial condyle articular surface with a radius of curvature, R₂, also for engagement with said insert,
   wherein R₁ is less than R₂.

In accordance with another aspect of the present invention, there is provided a tibial component for a prosthetic knee of a patient, including
(a) a non-articulating surface being couplable to a resected proximal end of a tibia of the patient; and
(b) a curved articular surface for engagement with a corresponding curved articular surface of an insert of the prosthetic knee,
   wherein the curved articular surface of the tibial component is adapted to facilitate rotation of the insert with respect to the tibial component.

Preferably, said curved articular surface of the tibial component is adapted to facilitate translation of the insert.

Preferably, the curved articular surface of the tibial component is convex.

In accordance with one aspect of the present invention, there is provided a insert for articulating a femoral component and a tibial component of a condylar surface prosthetic knee of a patient, including:
(a) a lateral articular surface for cooperative engagement with a lateral condyle articular surface of the femoral component;
(b) a medial articular surface for cooperative engagement with a medial condyle articular surface of the femoral component; and
(c) a curved articular surface for cooperative engagement with a corresponding curved articular surface of the tibial component.

In accordance with another aspect of the present invention, there is provided a method of fitting the above described prosthetic knee to a leg of a patient, including the steps of:
(a) coupling the femoral component to a distal end of a femur of the patient;
(b) coupling the tibial component to a proximal end of a tibia of the patient;
(c) arranging the insert so as to articulate with the femoral component and the tibial component,
   wherein the method preserves one or more of the cruciate ligaments of the patient and restores the mechanical axis of the leg.

### BRIEF DESCRIPTION OF THE DRAWINGS

Preferred embodiments of the present invention are hereafter described, by way of non-limiting example only, with reference to the accompanying drawing in which:

FIG. 1 is a diagrammatic illustration of an anterior view of a prosthetic knee in accordance with a preferred embodiment of the invention;

FIG. 2 is a diagrammatic illustration of the prosthetic knee shown in FIG. 1 fitted to a leg of a person and arranged in one condition of use;

FIG. 3 is a diagrammatic illustration of the prosthetic knee shown in FIG. 2 arranged in another condition of use;

FIG. 4 is a diagrammatic illustration of an anterior view of a femoral component of the prosthetic knee shown in FIG. 1;

FIG. 5 is a diagrammatic illustration of a lateral view of the femoral component shown in FIG. 4;

FIG. 5b is a diagrammatic illustration of a lateral view of an alternative femoral component;

FIG. 6 is a diagrammatic illustration of an inferior view of the femoral component shown in FIG. 4, showing the anticipated arrangement of the cruciate ligaments of the knee of the person to which the prosthetic knee is fitted;

FIG. 7 is a diagrammatic illustration of a posterior view of the femoral component shown in FIG. 4;

FIG. 8 is a diagrammatic illustration of a superior view of the femoral component shown in FIG. 4;

FIG. 9 is a diagrammatic illustration of an anterior view of a tibial component of the prosthetic knee shown in FIG. 1, showing the anticipated arrangement of the cruciate ligaments of the knee of the person to which the prosthetic knee is fitted;

FIG. 10 is a diagrammatic illustration of a superior view of the tibial component shown in FIG. 9;

FIG. 10b is a diagrammatic illustration of a superior view of an alternative tibial component;

FIG. 11 is a diagrammatic illustration of a lateral view of the tibial component shown in FIG. 9;

FIG. 12 is a diagrammatic illustration of an inferior view of the tibial component shown in FIG. 9;

FIG. 13 is a plan view of the proximal articular portion of a tibia of a person;

FIG. 14 is a perspective view of a resected tibia;

FIG. 15 is a perspective view of the of the resected tibia shown in FIG. 14 with the tibial component fitted;

FIG. 16 is a diagrammatic illustration of a perspective view of an insert of the prosthetic knee shown in FIG. 1;

FIG. 17 is a diagrammatic illustration of a cross-section of the insert shown in FIG. 16 on the line X-X;

FIG. 18 is a diagrammatic illustration of another cross-section of the insert shown in FIG. 16 on the line Y-Y;

FIG. 19 is a diagrammatic illustration of another cross-section of the insert shown in FIG. 16 on the line Z-Z;

FIG. 20 is a diagrammatic illustration of a superior view of the insert shown in FIG. 16;

FIG. 21 is a diagrammatic illustration of an inferior view of the insert shown in FIG. 16; and

FIG. 22 is a diagrammatic illustration of an anterior view of the prosthetic knee shown in FIG. 1 fitted to a leg of a person; and

FIG. 23 is a diagrammatic illustration of the prosthetic knee in accordance with another aspect of the present invention fitted to a leg of a person and arranged in one condition of use.

### DETAILED DESCRIPTION OF PREFERRED EMBODIMENTS OF THE INVENTION

As used herein, the following directional definitions apply. Anterior and posterior mean nearer the front and near the back of the body respectively. Thus, for the knee joint described herein, anterior refers to that portion of the knee that is nearer the front of the body when the leg is in an extended position. Proximal and distal respectively mean nearer to and further away from the root of the structure in question. For example, the distal end of the femur is the end of the femur that forms part of the knee joint and the proximal end of the femur is the end of the femur that forms part of the hip joint. Medial and lateral mean nearer to and further away from the sagittal plane respectively. The sagittal plane is the imaginary vertical plane that divides the body into left and right halves.

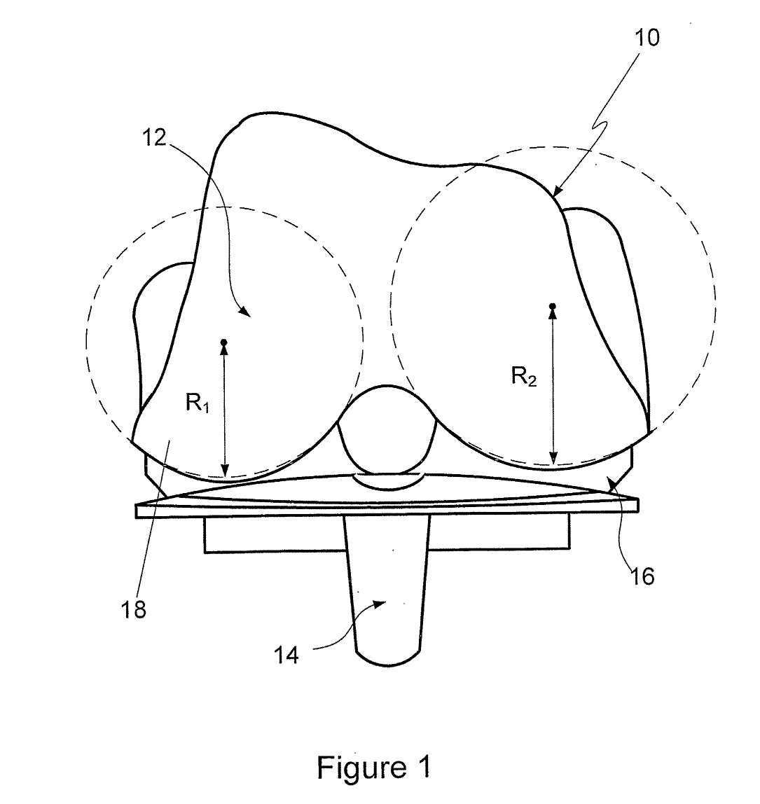

The prosthetic knee 10 shown in FIG. 1 advantageously restores normal functionality to the knee of a person. The prosthetic knee 10 utilises the tendons and ligaments surrounding the knee to hold the femoral and tibial portions of the joint together and to impart stability to the joint during movement.

The prosthetic knee 10 includes:
1. Femoral component 12;
2. Tibial component 14; and
3. Insert 16.

The femoral component 12 and the tibial component 14 articulate by way of the insert 16 arranged therebetween.

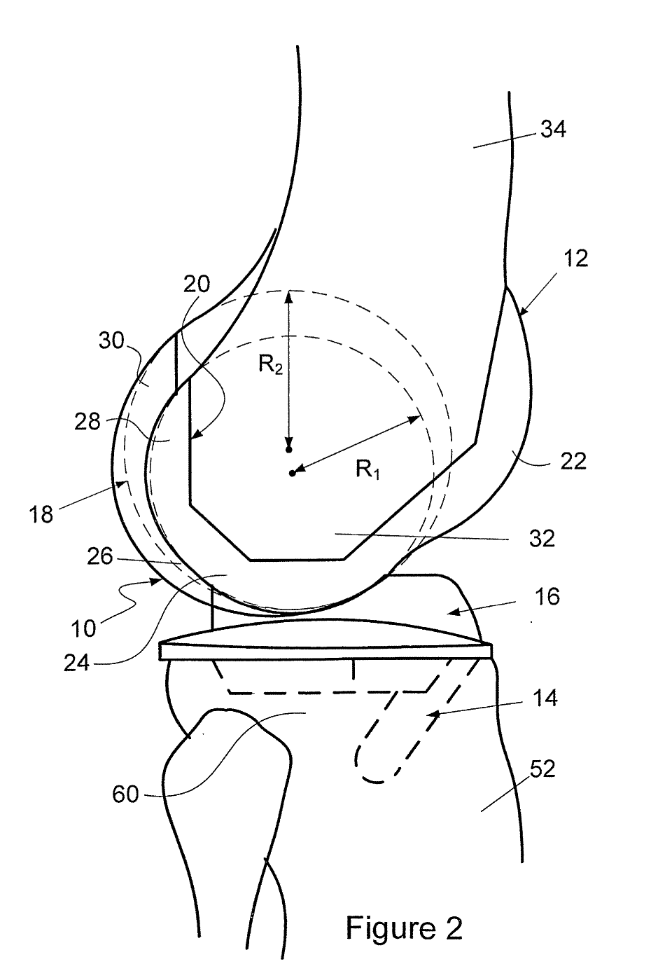

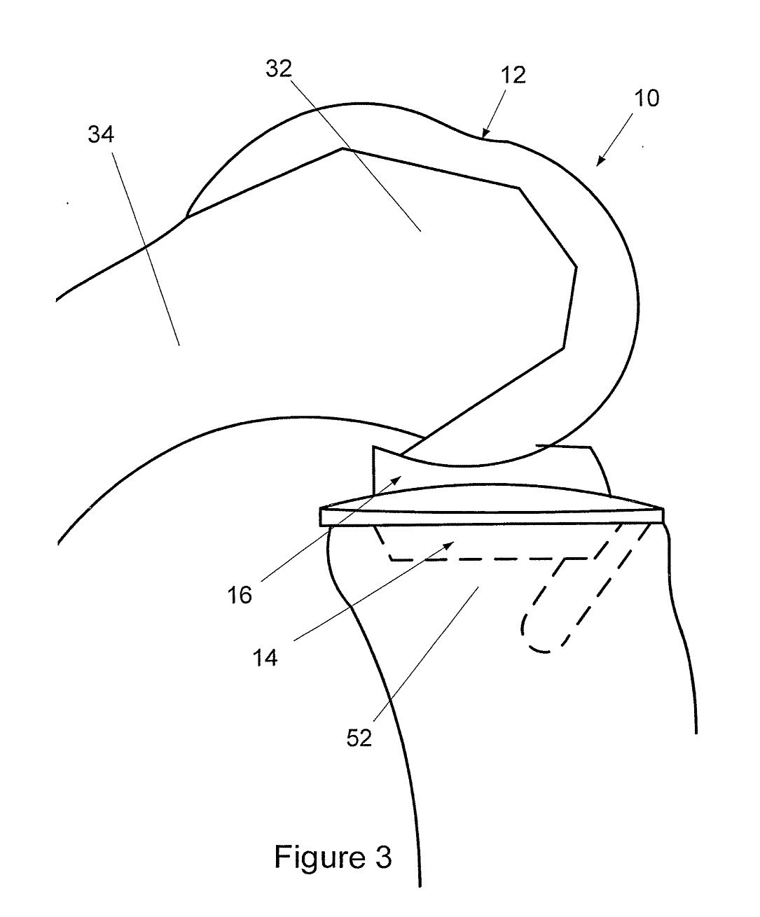

The prosthetic knee 10 shown in FIGS. 2 and 3 has been fitted to patient. The prosthetic knee 10 has a range of motion of five degrees hyperextension to approximately one hundred and thirty five degrees of flexion. At least part of the femoral component 12 remains in contact with the insert throughout the range of motion of the prosthetic knee 10. The prosthetic knee 10 utilises a sliding articulation to simulate femoral roll back. In flexion, for example, the insert 16 can translate posteriorly with respect to the tibial component 14, and the insert 16 can also rotate with respect to the tibial component 14 under bias from the femoral component 12.

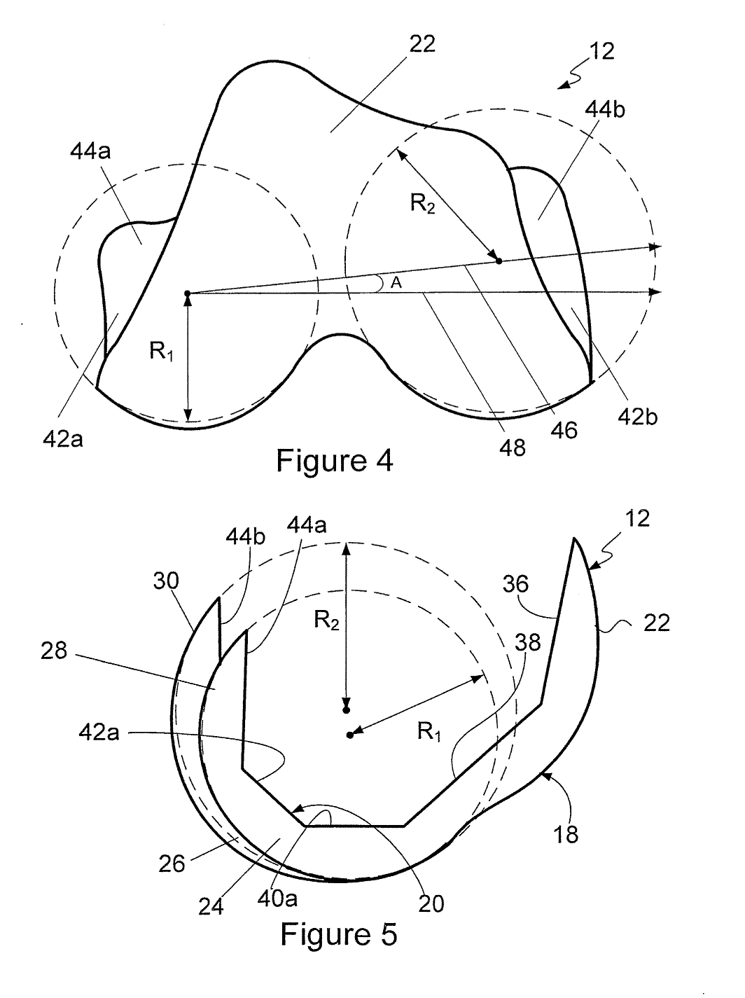

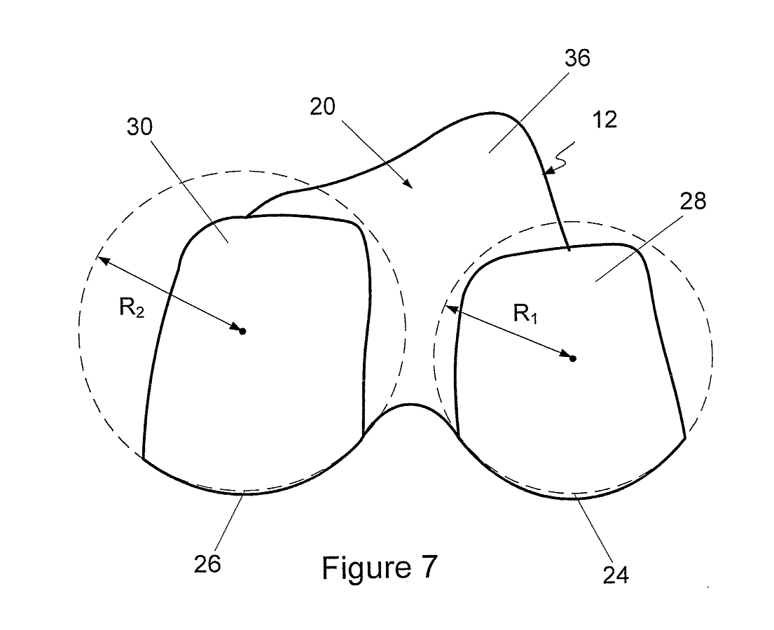

The femoral component 12 shown in FIGS. 4 to 8 includes an external articular surface 18 and a bone contacting non-articular internal surface 20.

The shape of the external articular surface 18 of the knee 10 is analogous to the distal bearing surfaces of a femur of a patient. The external articular surface 18 includes:
1. An anterior articular surface 22;
2. A distal lateral articular surface 24;
3. A distal medial articular surface 26;
4. A lateral posterior condyle articular surface 28; and
5. A medial posterior condyle articular surface 30.

The above surfaces 22,24,26,28,30 of the external articular surface 18 form a uniform curved surface that is shaped to operatively engage the insert 16.

The bone contacting non-articular internal surface 20 of the femoral component 12 is shaped to receive a resected distal end 32 of a femur 34 of a patient. The bone contacting non-articular internal surface 20 includes a plurality of chamfer surfaces. In use, surgeons make cuts in the distal end 32 of the femur 34 that correspond to the chamfer surfaces of the femoral component 12. Techniques for making these cuts are generally known in the art and are not discussed here in detail.

The bone contacting non-articular internal surface 20 includes a porous metal surface that promotes the growth of bone thereon. The bone contacting non-articular internal surface 20 alternatively includes any other suitable surface that promotes the growth of bone thereon. The bone contacting non-articular internal surface 20 may otherwise include a surface suitable for the use of orthopaedic bone cement for fixation of the femoral component 12 to the distal end 32 of the femur 34.

The non-articular surface 20 of the femoral component 12 includes the following components:
1. An anterior non-articular surface 36;
2. A distal anterior non-articular surface 38;
3. Two distal non-articular surfaces 40a,40b;
4. Two posterior non-articular surfaces 42a,42b; and
5. Two posterior non-articular surfaces 44a,44b.

The anterior non-articular surface 36 is generally flat and is shaped to receive and bear against an anterior section of the distal end 32 of the resected femur 34. The two distal non-articular surfaces 40a,40b are each generally flat and are shaped to receive and bear against respective extremities of the distal end 32 of the resected femur 34. The anterior non-articular surface 36 and the two distal non-articular surfaces 40a,40b are coupled together by the distal anterior non-articular surface 38.

The two posterior non-articular surfaces 44a,44b are generally flat and are shaped to receive and bear against respective posterior sections of the distal end 32 of the resected femur 34. Each one of two posterior non-articular surfaces 44a,44b is coupled to a corresponding one of the two distal non-articular surfaces 40a,40b by a respective one of the two posterior non-articular surfaces 42a,42b.

The distal lateral articular surface 24 and the lateral posterior condyle articular surface 28 form a section of a spherical surface, hereafter referred to as the lateral condyle articular surface, with a generally constant radius of curvature R₁. The distal medial articular surface 26 and the medial posterior condyle articular surface 30 also form a section of a spherical surface, hereafter referred to as the medial condyle articular surface, with a generally constant radius of curvature R₂, where R₁ is less than R₂. Those skilled in the relevant art will appreciate that a broad range of sizes R₁ and R₂ are applicable.

The lateral condyle articular surface preferably maintains the same arc of articulation as the medial condyle articular surface.

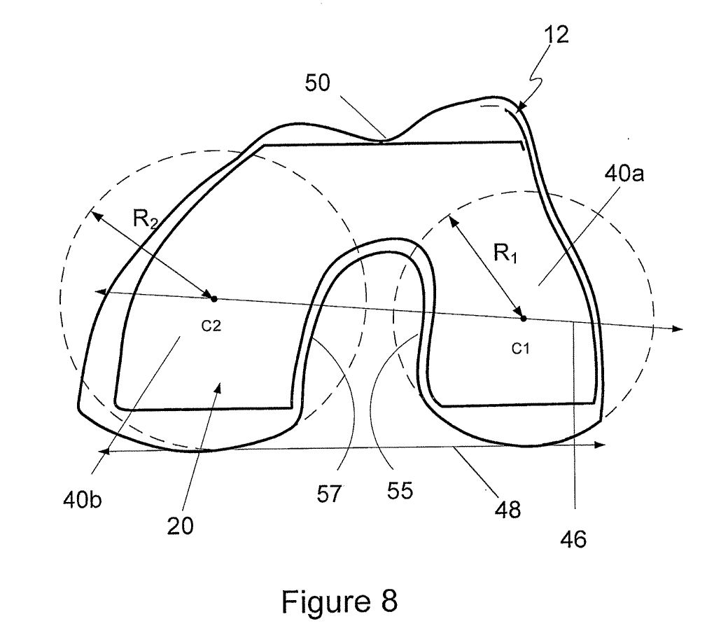

The epicondyle axis of the knee, shown in FIG. 8 as epicondyle line 46, extends between the centre of radius of curvature C₁, of the lateral condyle articular surface and the centre of radius of curvature C₂ of the medial condyle articular surface. The posterior condyle axis, shown in FIG. 8 as posterior condyle line 48, extends between a point on the lateral posterior condyle articular surface 28 and a corresponding point on the medial posterior condyle articular surface 30. The angle “A” formed between the epicondyle line 46 and the posterior condyle line 48 approximates the nonnal alignment of the knee. Angle “A” may three degrees, for example.

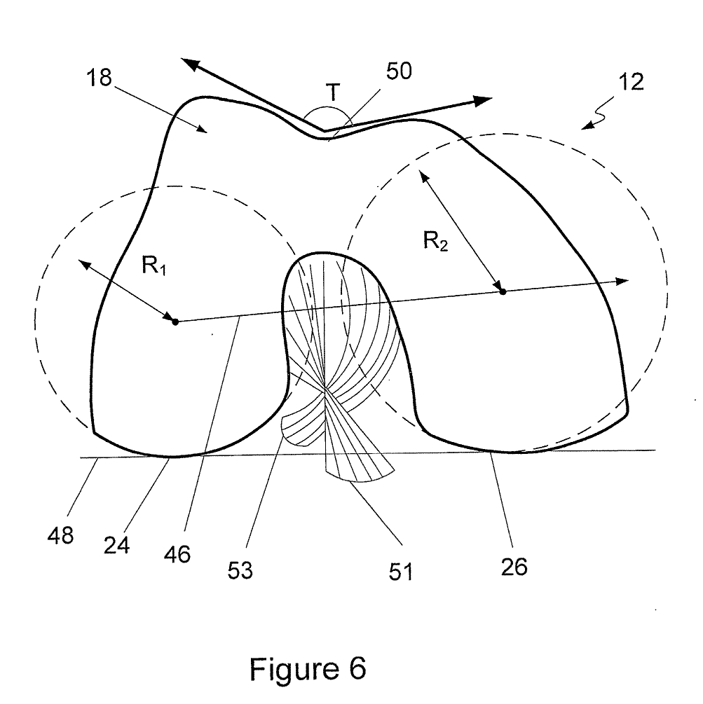

The anterior articular surface 22 includes a groove 50 for articulation with the replaced petella (not shown) of the prosthetic knee 10. The groove 50 is partially defined by two obtuse side walls. The side wall are preferably arranged at an angle of “T” degrees with respect to each other, as shown in FIG. 6. The angle “T” is preferably less than one hundred and fifty degrees. The groove 50 is positioned to run along the anatomical axis 94 of the femur 34 when the knee 10 is in extension.

The femoral component 12 is shaped to preserve the anterior 51 and posterior 53 ligaments of the knee of the patient, as illustrated in FIG. 6. The ligaments 51,53 extend through a gap defined by opposed side walls 55,57 of the lateral condyle articular surface and the medial condyle articular surface.

The femoral component 12 is made of any suitable biomaterial having the mechanical properties necessary to function as a human knee. The femoral component 12 is preferably made of titanium, titanium alloy, cobalt chrome alloy, stainless steel or ceramic, for example.

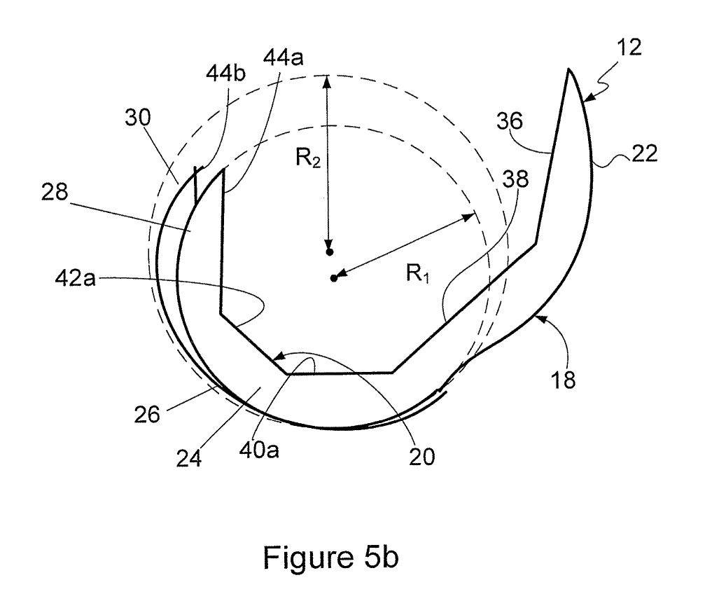

The alternative femoral component 12 shown in FIG. 5a includes a different radius of curvature in the saggital plane on the medial posterior condyle articular surface 30. The difference in radius of curvature, amongst other things, assists in locating the components of the prosthetic knee 10 the correct positions when fitting the prosthetic knee to the leg of the patient.

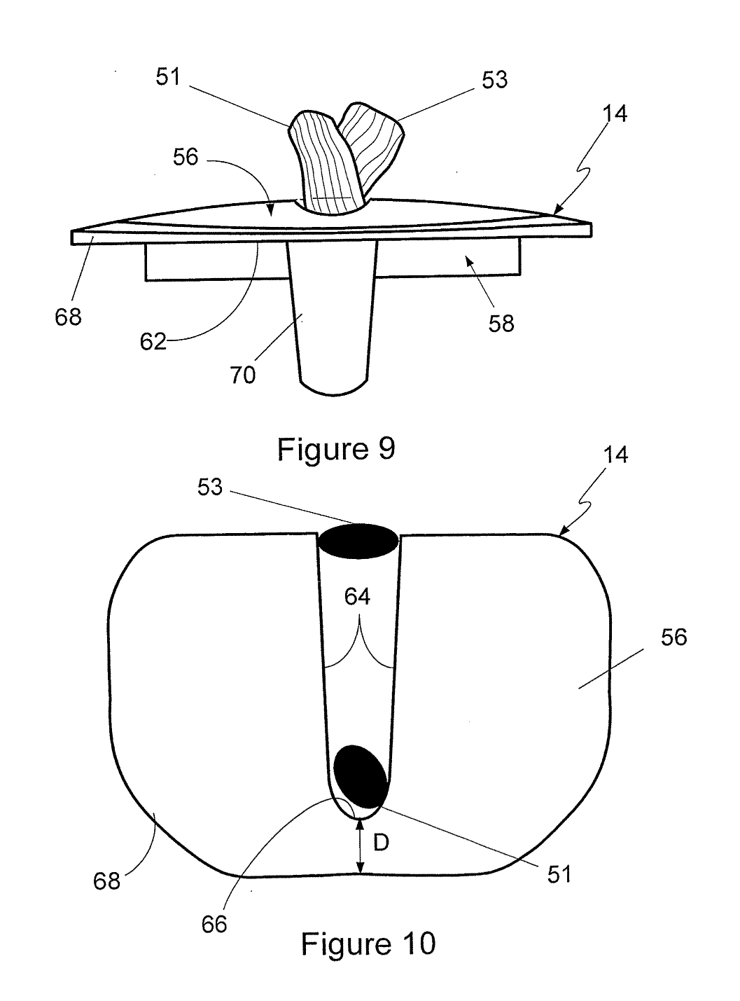

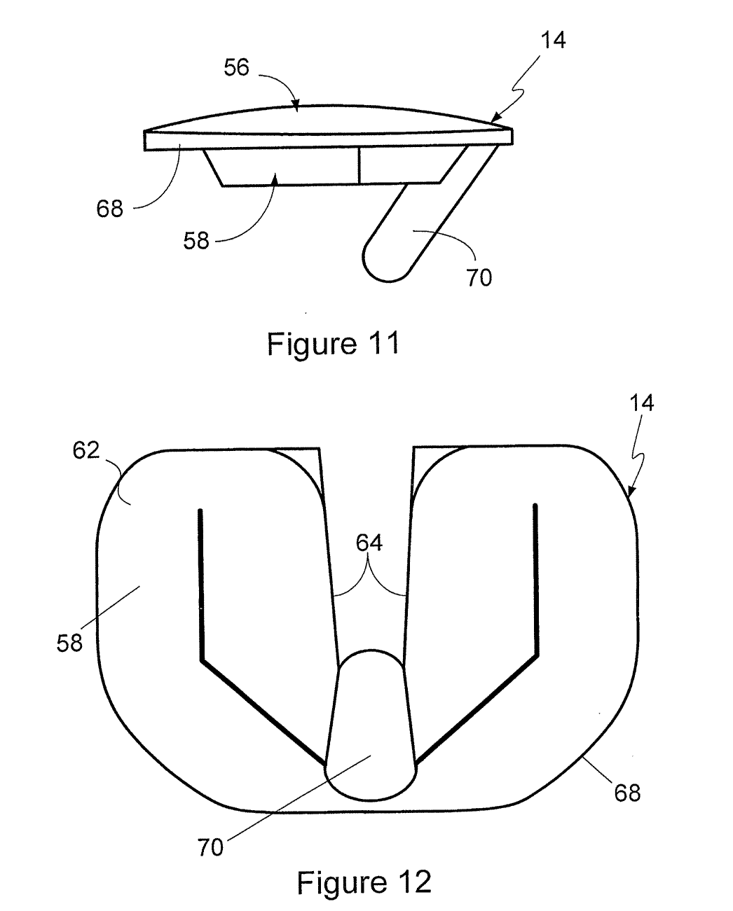

The tibial component 14 shown in FIGS. 9 to 12 includes an external articular surface 56 20 and a bone contacting surface 58. The tibial component 14 is made of any suitable biomaterial having the mechanical properties necessary to function as a human knee proximal tibial prosthesis. The tibial component 14 is preferably made of titanium, titanium alloy, cobalt chrome alloy, stainless steel or ceramic, for example.

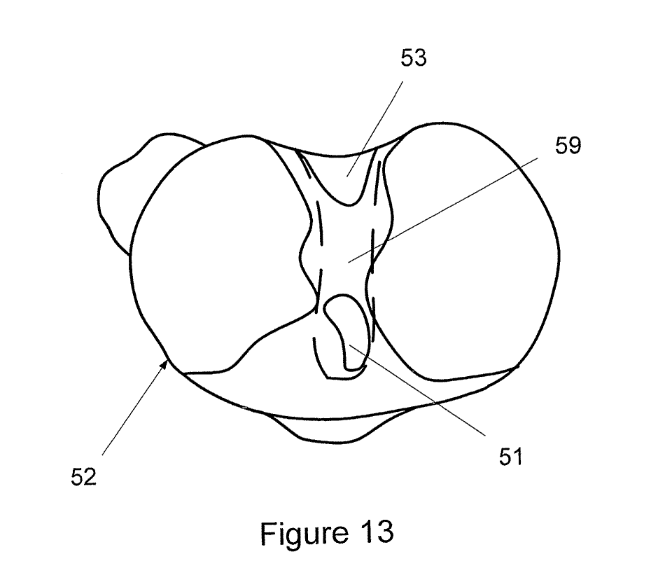

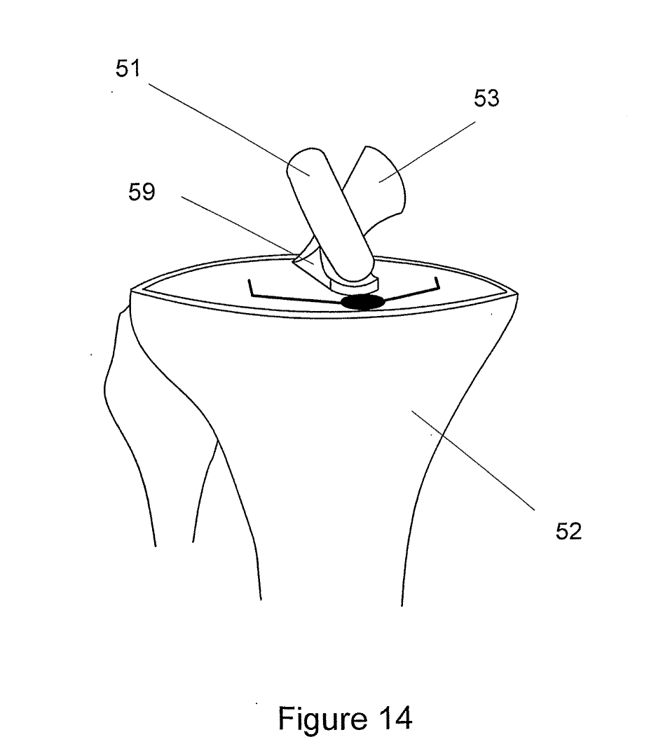

The inferior non-articular portion 58 of the tibial component 14 is shaped to receive a resected proximal end 60 of the tibia 52, as shown in FIGS. 2 and 3. The bone contacting non-articular internal surface 58 includes a plurality of chamfer surfaces. In use, surgeons make cuts in the distal end 60 of the tibia 52 that correspond to the chamfer surfaces of the tibial component 14, as shown in FIGS. 13 and 14. Techniques for making these cuts are generally known in the art and are not discussed here in detail. The non-articular portion 62 of the tibial component 14 that engages the proximal end 60 of the tibia 52 includes a porous metal surface 62, or any other like surface, to promote growth of bone thereon. The surface 62 alternatively includes a suitable surface suitable for the use of orthopaedic bone cement for fixation of the tibial component 14 to the proximal end 60 of the tibia 52.

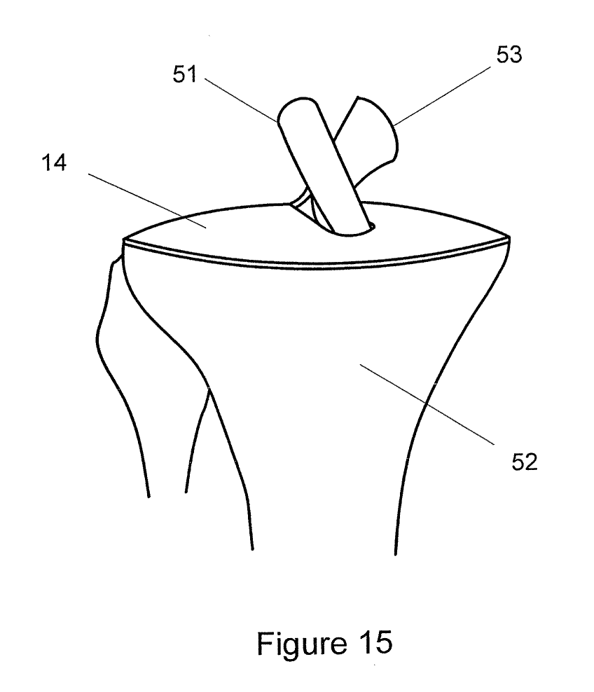

The tibial component 14 is includes a “U” shaped aperture that is at least partially defined by two spaced apart opposed medial surfaces 64 that extend from a curved anterior section 66 of the tibial component 14 towards the posterior of the tibial component 14. The aperture creates an opening between the superior external articular surface 56 and the inferior non-articular surface 58 of the tibial component 14. The aperture is shaped to receive a wedge of bone 59 preserved on the proximal end 60 of the tibia 52 that includes the anterior and posterior cruciate ligaments 51,53 of the leg of the patient. When so positioned, the cruciate ligaments extend from the tibia 52, through the aperture towards the insert 16, as shown in FIG. 15. The tibial component 14 thereby preserves the cruciate ligaments 51,53.

The outer peripheral edge surface 68 of the tibial component 14 are non-articular, non-bone contacting surfaces which are preferably continuous and connect to the two spaced apart opposed medial non-articular surfaces 64 posteromedially.

The tibial component also includes a tapered stem 70 coupled to the non-articular surface 58. The stem 70 is arranged in a position between the curved anterior non-articular section 66 and an anterior section of the non-articular surface 58. The stem 70 extends in a posteroinferior direction facilitating the intersection of a stemmed tibial component 14 in a cruciate preserving total knee replacement.

The articular surface 56 of the tibial component 14 is adapted to cooperatively engage and move with respect to the matched insert 16. The articular surface 56 retains the convex shape of the apical portion of a sphere, the radius of which approximates that of the distance from the knee to the ankle of the patient. The radius of curvature is, for example, greater than R₁ and R₂.

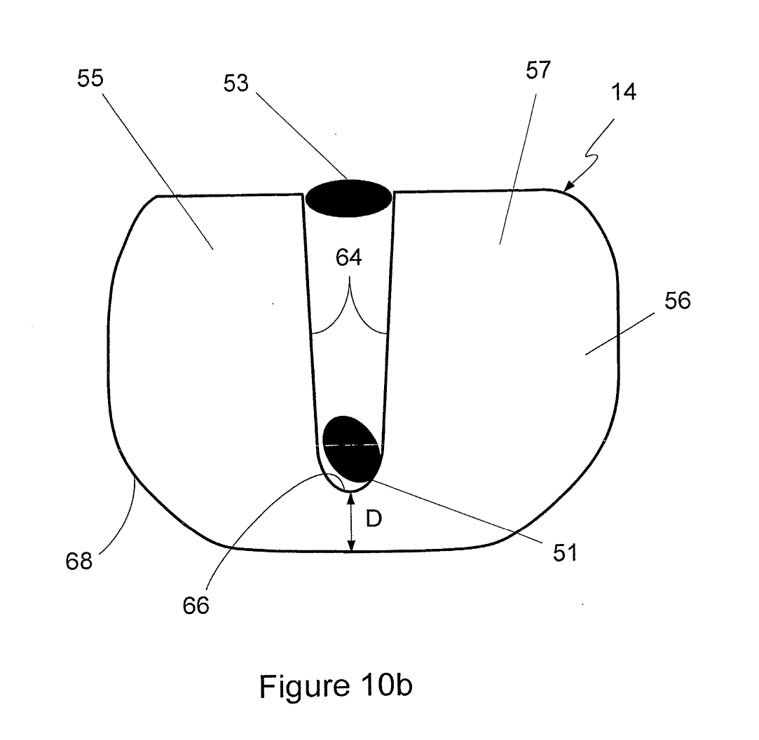

In the alternative tibial component 14 shown in FIG. 10b, the lateral articular surface 55 is less than the medial articular surface 57. In this embodiment, the articular surface 56 of the tibial component 14 is shaped to cover the entire contact area of the bone.

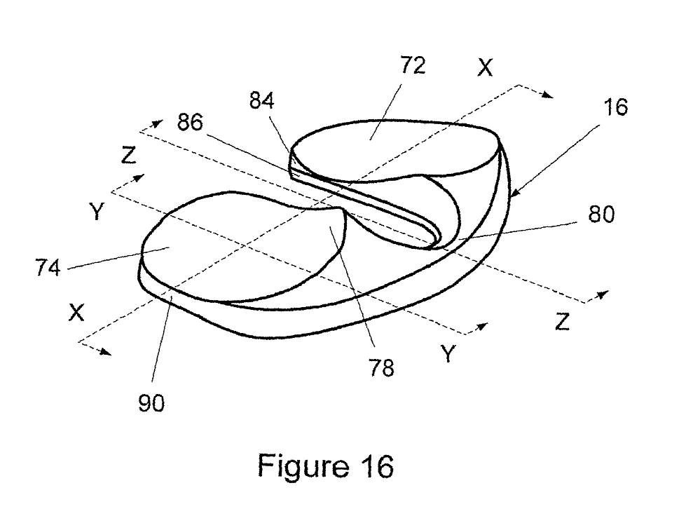

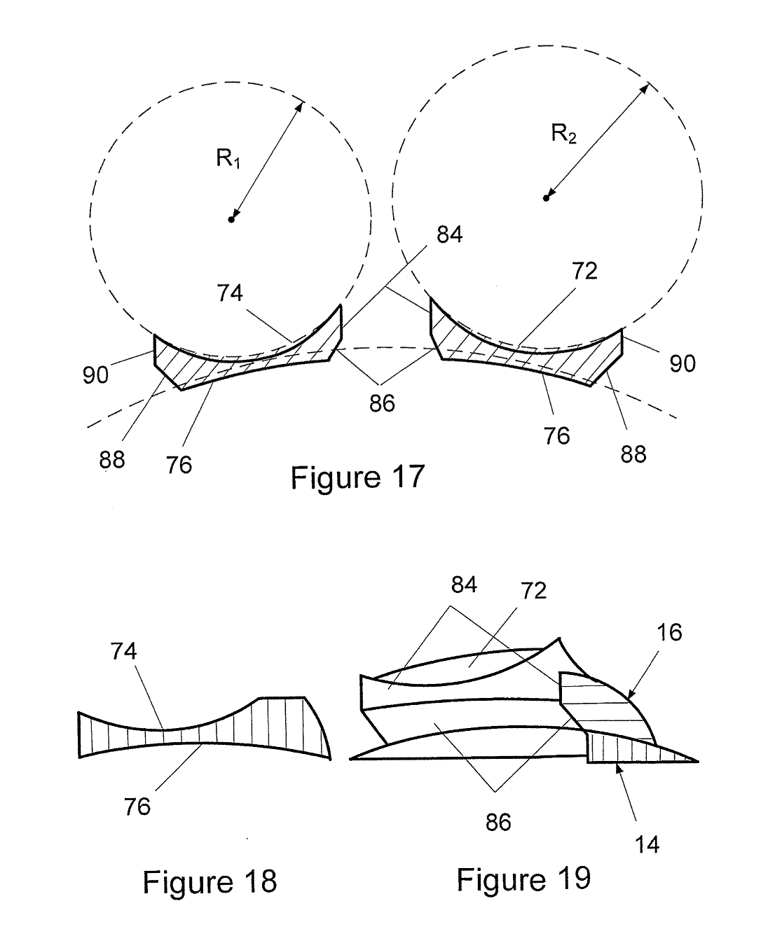

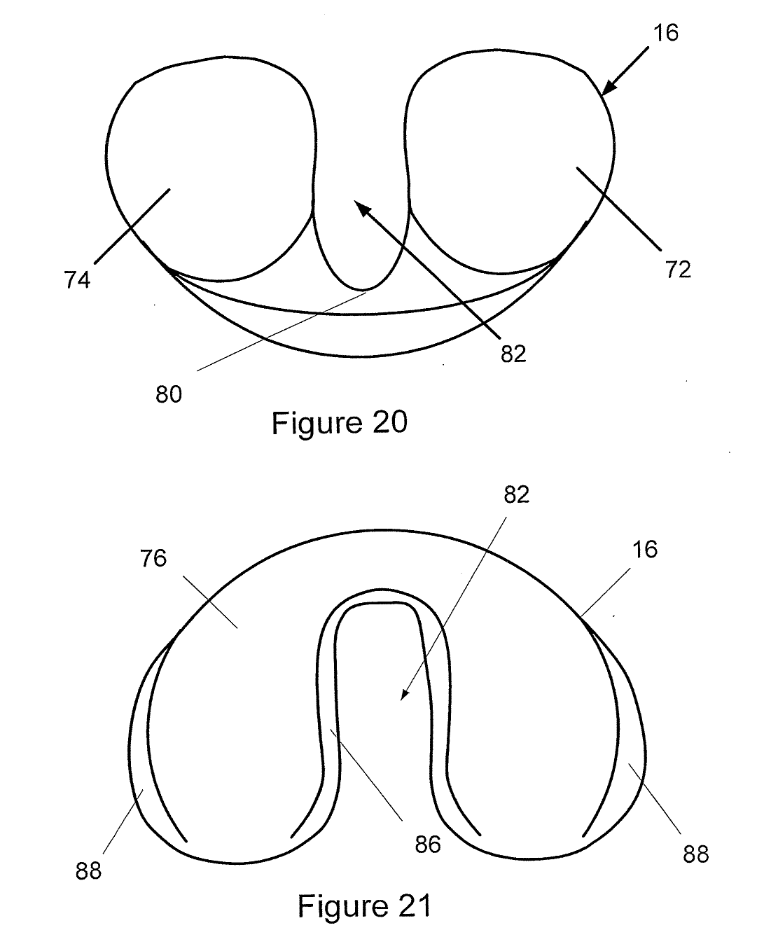

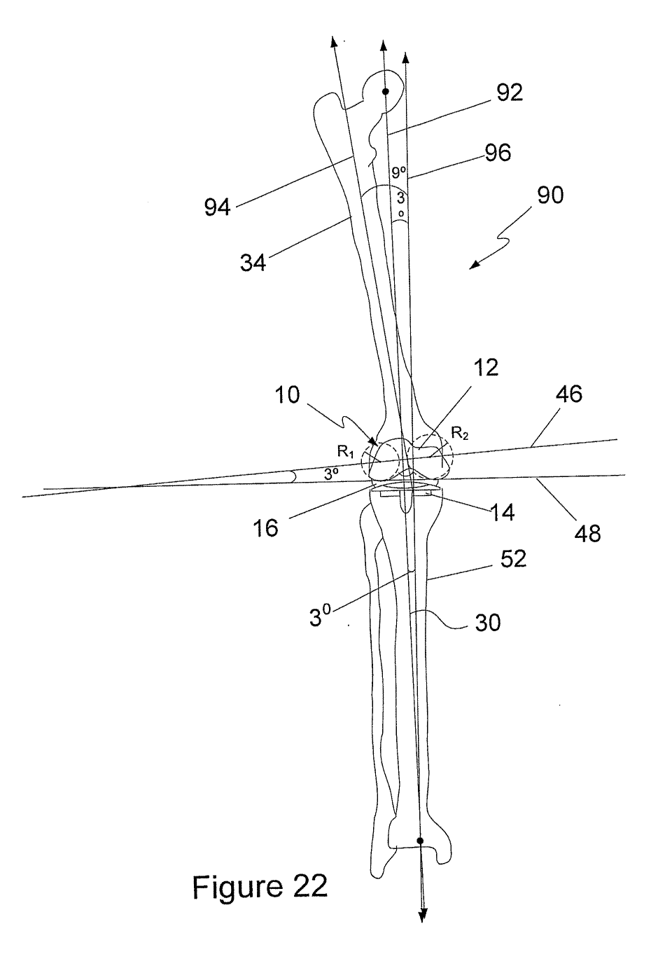

The prosthetic insert 16 shown in FIGS. 16 to 19 includes:
1. A medial superior articular surface 72;
2. A lateral superior articular surface 74; and
3. An inferior articular surface 76.

The medial superior articular surface 72 is adapted to cooperatively engage the medial articular surfaces 26,30 of the femoral component 12. The lateral superior articular surface 74 is adapted to cooperatively engage the lateral articular surfaces 24, 28 of the femoral component 12. The inferior articular surface 76 is generally concave and is shaped to receive the convex articular surface 56 of the tibial component 14.

The articular surface 74 has a raised section in the anteromedial portion of the surface 78. The raised section increases the contact area with the femoral component 12 whilst avoiding soft tissue impingement.

The insert includes a non-articular surface 80 arranged between the medial superior articular surface 72 and the lateral superior articular surface 74. The arrangement of these surfaces 72,74,80 defines a central opening 82 in the insert 16 that accommodates the cruciate ligaments 51,53 of the patient.

The insert also includes:
1. Two central superior non-articular surfaces 84;
2. Two central inferior non-articular surfaces 86; and
3. Two lateral inferior non-articular surfaces 88.

The two central inferior non-articular surfaces 86 are preferably generally flat and are adapted to preserve the cruciate ligaments. One of the two central superior non-articular surfaces 84 is arranged side by side with one of the two central inferior non-articular surfaces 86. The other ones of the respective surfaces 84,86 are also arranged with respect to each other in the same manner. The surfaces 84,86 of each pair of surfaces are disposed at an obtuse angle with respect to each other to avoid soft tissue impingement

The two central inferior non-articular surfaces 86 extend inferiorly from their respective central superior non-articular surfaces 84 to abut the articular surface 76.

The two lateral inferior non-articular surfaces 88 extend inferiorly to abut the articular surface 76. The lateral inferior non-articular surfaces 88 extend superiorly to abut the non-articular surface 90.

The insert 16 is made of any suitable biomaterial having the mechanical properties necessary to function as a human knee prosthetic insert. The insert 16 is preferably made of titanium, titanium alloy, cobalt chrome alloy, stainless steel, ceramic or polyethylene, for example.

The right leg 90 of the patient shown in FIG. 19 is arranged in an extended condition of use. The right leg 46 includes:
1. A femur 34;
2. Prosthetic femoral component 12;
3. Prosthetic insert component 16;
4. Prosthetic tibial component 14; and
5. Tibia 52.

The drawing of the leg 46 indicates:
1. The mechanical axis of the leg, shown as line 92;
2. The anatomical axis of the femur, shown as line 94; and
3. A vertical axis, shown as line 96.

In a normal leg 90, the mechanical axis of the leg 92 is typically arranged an angle of three degrees to the vertical axis 96. Further, the anatomical axis of the femur 94 is typically arranged at an angle of nine degrees to the vertical axis 96. When the prosthetic knee 10 is fitted to a patient, the angle between the epicondyle axis 46 and the posterior condyle axis 48 is three degrees and the posterior condyle axis 48 (also referred to as distal the femoral joint line of the prosthetic knee) aligns the anatomical axis of the femur 94 at substantially nine degrees to the vertical axis 96. In this arrangement, the mechanical axis of the leg 92 is disposed at an angle of three degrees to the vertical axis 96.

The insert 16 and tibial component 14 are positioned at three degrees of varus in relation to the mechanical axis 92. Those skilled in the relevant art will appreciate that a number of angles may be employed by the present invention.

The concave lateral and the medial superior articular surfaces 74,72 of the insert 16 are shaped to receive respective ones of the lateral and medial condyle articular surfaces. Movement of the articulating surface 18 of the femoral component 12 is controlled by the shape and configuration of the concave lateral and the medial superior articular surfaces 74,72 of the insert 16. The shape of the articulating surface 18 of the femoral component 12 and the corresponding lateral and medial superior articulating surfaces 72,74 of the insert 16 permit sliding articulation to simulate femoral roll back of the prosthetic knee 10. That is, the components 12,14,16 permit posterior/anterior translation of the femoral articular surface 18 with respect to the tibial articular surface 56 during flexion/extension. The concave shape of the lateral and the medial superior articular surfaces 74,72 of the insert 16 assists in maintaining a layer of lubrication between the articular surfaces of the femoral component 12 and the insert 16.

The concave inferior articular surface 76 of the insert is shaped to receive the convex articular surface 56 of the tibial component 14. The arrangement of these two articulating surfaces 76,56 facilitates a wide range of movement between the respective components 16,14. For example, the convex shape of the articular surface 56 of the tibial component 14 facilitates sliding articulation with the inferior concave articulating surface 76 of the insert. The tibial component 14 can there by rotate with respect to the inset during flexion/extension of the prosthetic knee 10. The concave and convex articular surfaces 76, 56 also assist in maintaining a layer of lubricant between the abutting portions of the components.

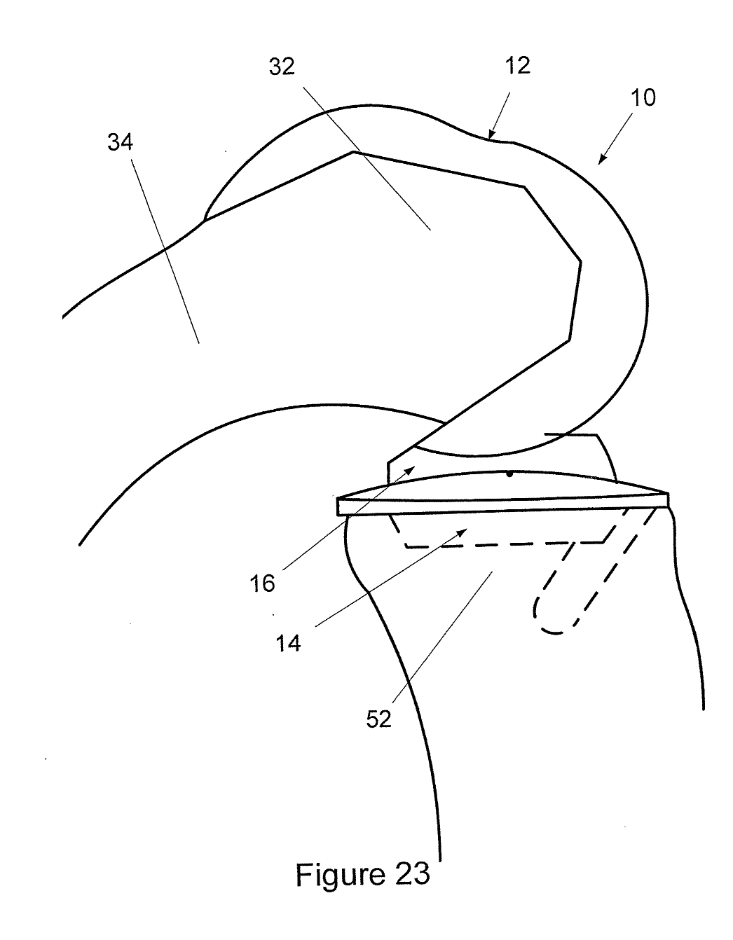

The prosthetic knee 10 shown in FIG. 23 functions in an analogous manner to that of the prosthetic knee 10 shown in FIGS. 1 to 3. However, a posterior section of the insert 16 has been reshaped to facilitate easier installation of the prosthetic knee.

While we have shown and described specific embodiments of the present invention, further modifications and improvements will occur to those skilled in the art. We desire it to be understood, therefore, that this invention is not limited to the particular forms shown and we intend in the append claims to cover all modifications that do not depart from the spirit and scope of this invention.

### LIST OF PARTS

Prosthetic knee 10

Femoral component 12

Tibial component 14

Insert 16

External articular surface 18

Bone contacting non-articular internal surface 20

Anterior articular surface 22

Distal lateral articular surface 24

Distal medial articular surface 26

Lateral posterior condyle articular surface 28

Medial posterior condyle articular surface 30

Distal end 32 of the femur 34

Anterior non-articular surface 36

Distal anterior non-articular surface 38, 40a,40b

Posterior non-articular surface 42a42b,44a,44b

Epicondyle line 46

Posterior condyle line 48

Groove 50

Anterior cruciate ligament 51

Posterior cruciate ligament 53

Tibia 52

Lateral articular surface 55

External articular surface 56

Medial articular surface 57

Bone contacting surface 58

Inferior non-articular portion of the tibial component 58

Wedge of bone 59

Proximal end 60 of the tibia 52

Porous metal surface 62

Medial non-articular surfaces 64

Curved anterior non-articular section 66 of the tibial component 14

Outer peripheral edge surface 68 of the tibial component 14

Medial superior articular surface 72

Lateral superior articular surface 74

Inferior articular surface 76

Anteromedial portion of the surface 78

Non-articular surface 80

Central opening 82

Central superior non-articular surfaces 84

Central inferior non-articular surfaces 86

Lateral inferior non-articular surfaces 88

Right leg of a person 90

Mechanical axis of the leg 92

Anatomical axis of the femur 94

Vertical axis 96

1. A prosthetic knee for replacing a knee of a patient including:
(a) a femoral component being couplable to a resected distal end of a femur of the patient;
(b) a tibial component being couplable to a resected proximal end of a tibia of the patient; and
(c) an insert wherein the femoral component and the tibial component articulate by way of the insert arrangable therebetween and a curved articular surface of the insert is adapted to pivot with respect to a corresponding curved articular surface of the tibial component.

2. The prosthetic knee according to claim 1, wherein said curved articular surface of the tibial component is adapted to facilitate translation of the insert with respect to the tibial component.

3. The prosthetic knee according to claim 1, wherein the curved articular surface of the tibial component is convex and the curved articular surface of the insert is concave, and said concave articular surface is adapted to at least partially receive said convex articular surface.

4. The prosthetic knee according to claim 1, wherein the femoral component includes a lateral condyle articular surface and a medial condyle articular surface for respectively articulating with a lateral articular surface and a medial articular surface of the insert.

5. The prosthetic knee according to claim 4, wherein the lateral articular surface and the medial articular surface of the insert are both concave and are shaped to at least partially receive respective ones of the lateral condyle articular surface and the medial condyle articular surface of the femoral component.

6. The prosthetic knee according to claim 4, wherein the lateral condyle articular surface has a radius of curvature, R₁, and the medial condyle articular surface has a radius of curvature, R₁, where R₁ is less than R₂.

7. The prosthetic knee according to claim 6, wherein the lateral condyle articular surface is a section of a substantially spherical surface having a radius of curvature of R₁ and the medial condyle articular surface is a section of a substantially spherical surface having a radius of curvature of R₂.

8. The prosthetic knee according to claim 4, wherein the articular surface of the femoral component and corresponding articular surface of the insert are adapted to effect femoral roll back by way of sliding articulation.

9. The prosthetic knee according to claim 1, wherein the tibial component includes an aperture through which one or more of cruciate ligaments of the knee of the patient can extend from the tibia towards the femur.

10. The prosthetic knee according to claim 9, wherein said aperture is shaped to at least partially receive a portion of the proximal end of the tibia including said one or more cruciate ligaments.

11. The prosthetic knee according to claim 9, wherein said aperture generally “U” shaped and is at least partially defined by two spaced apart opposed medial surfaces that extend posterioraly from an anterior section of the tibial component.

12. The prosthetic knee according to claim 11, wherein the tibial component includes a stem for coupling the prosthetic knee to the tibia, said stem extending posteroinferiorially from a non-articulating inferior section of said anterior section of the tibial component.

13. The prosthetic knee according to claim 1 wherein the insert includes an aperture through which one or more of the cruciate ligaments can extend from the tibia towards the femur.

14. The prosthetic knee according to claim 13, wherein said aperture generally “U” shaped and is at least partially defined by two spaced apart opposed medial surfaces that extend posterioraly from an anterior section of the insert.

15. The prosthetic knee according to claim 1, wherein the femoral component includes a groove at least partially defined by side walls arranged at an angle ranging from less than less than one hundred and seventy degrees in extension of the prosthetic knee to less than one hundred and forty degrees in flexion of the prosthetic knee.

16. The prosthetic knee according to claim 1, wherein a distal femoral joint line of the prosthetic knee aligns the anatomical axis of the femur at substantially nine degrees to a vertical axis.

17. The prosthetic knee according to claim 1, wherein an angle between an epicondyle axis of the femoral component and a distal femoral joint line of the femoral component is substantially three degrees.

18. The prosthetic knee according to claim 1, wherein the difference between R₁ and R₂ facilitates one to five degrees of external rotation in flexion with respect to an epicondyle axis and a posterior condyle axis of the prosthetic knee.

19. The prosthetic knee according to claim 1, wherein the knee has a range of motion of five degrees hyperextension and one hundred and thirty five degrees flexion.

20. The prosthetic knee according to claim 1, wherein the prosthetic knee utilises the tendons and ligaments surrounding the knee to hold the femoral and tibial components together and to impart stability to the knee during movement.

21. A femoral component for a condylar surface prosthetic knee of a patient, including:
(a) a non-articulating surface being couplable to a resected distal end of a femur of the patient;
(b) a lateral condyle articular surface with a radius of curvature, R₁, for engagement with an insert of the prosthetic knee; and
(c) a medial condyle articular surface with a radius of curvature, R₂, also for engagement with said insert, wherein R₁ is less than R₂.

22. The femoral component according to claim 21, wherein the lateral condyle articular surface is a section of a substantially spherical surface having a radius of curvature of R₁ and the medial condyle articular surface is a section of a substantially spherical surface having a radius of curvature of R₂.

23. The femoral component according to claim 21, wherein the lateral condyle articular surface maintains substantially the same arc of articulation as the medial condyle articular surface.

24. The femoral component according to claim 21, including a groove at least partially defined by side walls arranged at an angle ranging from less than less than one hundred and seventy degrees in extension of the prosthetic knee to less than one hundred and forty degrees in flexion of the prosthetic knee.

25. The femoral component according to claim 21, wherein the difference between R1 and R2 facilitates one to five degrees of external rotation in flexion with respect to epicondyle axis and posterior condyle axis of the prosthetic knee.

26. The femoral component according to claim 21, wherein an epicondyle axis of the prosthetic knee is transverse to a posterior condyle axis of the prosthetic knee.

27. A tibial component for a prosthetic knee of a patient, including
(a) a non-articulating surface being couplable to a resected proximal end of a tibia of the patient; and
(b) a curved articular surface for engagement with a corresponding curved articular surface of an insert of the prosthetic knee, wherein the curved articular surface of the tibial component is adapted to facilitate rotation of the insert with respect to the tibial component.

28. The tibial component according to claim 27, wherein said curved articular surface of the tibial component is adapted to facilitate translation of the insert.

29. The tibial component according to claim 27, wherein the curved articular surface of the tibial component is convex.

30. The tibial component according to claim 27, including an aperture through which the one or more of the cruciate ligaments can extend from the tibia towards the femur.

31. The tibial component according to claim 30, wherein said aperture is shaped to at least partially receive a portion of the proximal end of the tibia of the patient that includes said one or more cruciate ligaments.

32. The tibial component according to claim 30, wherein said aperture is generally “U” shaped and is at least partially defined by two spaced apart opposed medial surfaces that extend posterioraly from an anterior section of the tibial component.

33. The tibial component according to claim 32, wherein the tibial component includes a stem for coupling the prosthetic knee to the tibia, said stem extending posteroinferiorially from a non-articulating inferior section of said anterior section of the tibial component.

34. The tibial component according to claim 27, wherein the nonarticulating surface includes a flange for engaging the proximal end of the tibia.

35. An insert for articulating a femoral component and a tibial component of a condylar surface prosthetic knee of a patient, including:
(a) a lateral articular surface for cooperative engagement with a lateral condyle articular surface of the femoral component;
(b) a medial articular surface for cooperative engagement with a medial condyle articular surface of the femoral component; and
(d) a curved articular surface for cooperative engagement with a corresponding curved articular surface of the tibial component.

36. The insert according to claim 35, including an aperture through which one or more cruciate ligaments can extend from the tibia to the femur.

37. The insert according to claim 36, wherein said aperture separates the medial articular surface and the lateral articular surface of the insert.

38. The insert according to claim 35, wherein the lateral articular surface and the medial articular surface facilitates sliding articulation with the lateral condyle articular surface and the medial condyle articular surface of the femoral component.

39. The insert according to claim 35, wherein the curved articular surface of the insert is adapted to pivot with respect to the corresponding curved articular surface of the tibial component.

40. The insert according to claim 35, wherein said curved articular surface of the insert is adapted to facilitate translation of the insert with respect to the tibial component.

41. The insert according to claim 35, wherein the curved articular surface of the insert component is concave.

42. A method of fitting the prosthetic knee claimed in claim 1 to a leg of a patient including the steps of:
(a) coupling the femoral component to a distal end of a femur of the patient;
(b) coupling the tibial component to a proximal end of a tibia of the patient; (c) arranging the insert so as to articulate with the femoral component and the tibial component wherein the method preserves one or more of the cruciate ligaments of the patient and restores the mechanical axis of the leg.
